(19) United States
(12) Patent Application Publication
(10) Pub. No.: US 2018/0235795 A1
(43) Pub. Date: Aug. 23, 2018

(54) PRESCRIBED MOTION BRACE

(71) Applicants: Laurel Kuxhaus, Potsdam, NY (US); Brian Davis, Wynantskill, NY (US); Anthony D. Anderson II, Potsdam, NY (US); Megan Curry, Syracuse, NY (US); Catherine Pelton, Auburn, NH (US); Christopher Robinson, Queensbury, NY (US); Margaret Shea, Potsdam, NY (US)

(72) Inventors: Laurel Kuxhaus, Potsdam, NY (US); Brian Davis, Wynantskill, NY (US); Anthony D. Anderson II, Potsdam, NY (US); Megan Curry, Syracuse, NY (US); Catherine Pelton, Auburn, NH (US); Christopher Robinson, Queensbury, NY (US); Margaret Shea, Potsdam, NY (US)

(73) Assignee: Clarkson University, Potsdam, NY (US)

(21) Appl. No.: 15/897,681

(22) Filed: Feb. 15, 2018

Related U.S. Application Data
(60) Provisional application No. 62/460,174, filed on Feb. 17, 2017.

(51) Int. Cl. A61F 5/01

(52) U.S. Cl. CPC A61F 5/013; A61F 2005/0137; A61F 2005/0165; A61F 2005/0148

(57) ABSTRACT

A brace, comprising: (i) a palmar piece configured to conform to the palmar side of a user's wrist, comprising an opening sized for the user's thumb, the palmer piece further comprising a first pin extending outwardly from an outer surface of the palmar piece in a first direction, further comprising a second pin extending outwardly from the outer surface in a first direction, wherein the first and second direction are opposite one another; and (ii) a forearm piece configured to conform to the user's forearm, comprising a first pin opening sized to receive the first pin, further comprising an elongated slot configured to receive the second pin in a sliding relationship; wherein the palmar piece is moveable relative to the forearm piece such that the user can move the wrist along the user's dart thrower's plane.

### CROSS-REFERENCE TO RELATED APPLICATIONS

The present application claims priority to U.S. Provisional Application No. 62/460,174, filed on Feb. 17, 2017, and entitled “Prescribed Motion Brace: Daily Advanced Range of Motion Therapy for Maximizing Function,” the entire contents of which are hereby incorporated by reference in its entirety.

### GOVERNMENT FUNDING

This invention was made with Government support under the National Science Foundation Award GARDE-1510367. The United States Government has certain rights in the invention.

### FIELD OF THE INVENTION

The present disclosure is directed generally to a limited mobility brace providing user support and comfort while allowing selective motion.

### BACKGROUND

Injury to the scapho-lunate interosseous ligament (SLIL), the primary stabilizer of the wrist, is one of the most common wrist traumas. Tears or defects to this ligament occur in 29% of the population (a cadaver study) and, according to the U.S. Census Bureau, wrist injuries are accountable for 10 days of work loss each year per worker sustaining an injury. This injury affects people of all ages and often requires surgery. Minor injury, such as a ligament tear, often results from overuse of the wrist, while a traumatic dislocation of the ligament often occurs from falling on the outstretched hand.

Injuries to the SL ligament can be treated by restricting motion or by surgical repair. Typical recovery from the SL ligament surgery includes 6-8 weeks of immobilization. It is possible to shorten the immobilization time with a limited motion brace that restricts motion to, for example, the dart thrower's plane (DTP), although other planes are possible based on the injury and/or prescribed treatment. Limited motion braces promote bone growth, healing of associated ligaments, and joint mobility. Movements in the DTP minimize the risk of reinjuring the SL ligament via strain while allowing the patient to regain limited mobilization earlier than with traditional motionless braces, maximizing function during recovery. This improves quality of life and shortens total recovery time. Furthermore, post-injury joint activity promotes blood flow and contributes to a faster recovery time. The dart thrower's motion (DTM) is defined by wrist motion starting at 30° extension with 10° radial deviation and moves through 30° flexion with 10° ulnar deviation.

Current DTP wrist braces are not commercially available. The nature of the slightly oblique DTP and the establishment of a patient-specific axis of rotation poses a significant design challenge. Most current braces are rigid and allow no movement, while some allow a reduced total range of motion of the joint. Existing braces do not create planes of allowable motion for the joint. Indeed, it is challenging to design a wrist brace that allows movement only in the DTP while optimizing cost and manufacturing efficiency.

Accordingly, there is a continued need for a wrist brace that restricts motion to the DTP while delivering accurate, smooth movement, and which is affordable and easy to don and doff.

### SUMMARY OF THE INVENTION

The present disclosure is directed to a wrist brace system. In one embodiment, the system is a wrist brace that allows movement only in the dart thrower's plane (DTP). The wrist brace design is more user friendly and comfortable than existing designs. In function, the brace restricts motion to the DTP while delivering accurate, smooth movement. The brace does not hinder the healing process of the SL ligament or the incision area.

According to one aspect of the present disclosure is a wrist brace. The wrist brace comprises: (i) a palmar piece configured to conform to the palmar side of a user's wrist, the palmar piece comprising an opening sized for the user's thumb to pass there through, the palmer piece further comprising a first pin extending outwardly from an outer surface of the palmar piece in a first direction, the palmar piece further comprising a second pin extending outwardly from the outer surface of the palmar piece in a first direction, wherein the first direction and the second direction are opposite one another; and (ii) a forearm piece configured to conform to the user's forearm, the forearm piece, the forearm piece comprising a first pin opening sized to receive the first pin, the forearm piece further comprising an elongated slot configured to receive the second pin in a sliding relationship, wherein the elongated slot extends along an axis perpendicular to the user's arm in the brace; wherein the palmar piece is moveable relative to the forearm piece when the first pin is installed in the first pin opening and the second pin is installed in the elongated slot, such that the user can move the wrist along the user's dart thrower's plane.

According to an embodiment, the palmar piece is configured such that the first pin is positioned about the user's scaphoid tubercle.

According to an embodiment, the forearm piece is configured such that the second pin is positioned above the user's 4^{th} metacarpal.

According to an embodiment, a diameter of the first pin opening is larger than a diameter of the first pin.

According to an embodiment, the diameter of the first pin opening is approximately 1.5 mm larger than the diameter of the first pin.

According to an embodiment, at least a portion of an outer edge of the brace is thicker than an inner portion of the brace.

According to an embodiment, at least a portion of an outer edge of the thumb opening is thicker than an inner portion of the brace

According to an embodiment, at least a portion of the forearm piece comprises meshing.

According to an embodiment, the forearm piece comprises two or more slots configured to receive an attachment mechanism around the dorsal portion of the user's forearm.

According to an embodiment, further comprising a stopping mechanism.

According to an aspect is a brace for a user. The brace includes: (i) a palmar piece configured to conform to the palmar side of a user's wrist, the palmar piece comprising an opening sized for the user's thumb to pass there through, the palmer piece further comprising a first pin extending outwardly from an outer surface of the palmar piece in a first direction, the palmar piece further comprising a second pin extending outwardly from the outer surface of the palmar piece in a first direction, wherein the first direction and the second direction are opposite one another, further wherein the palmar piece is configured such that the first pin is positioned about the user's scaphoid tubercle; and (ii) a forearm piece configured to conform to the user's forearm, the forearm piece comprising a first pin opening sized to receive the first pin, the forearm piece further comprising an elongated slot configured to receive the second pin in a sliding relationship, wherein the elongated slot extends along an axis perpendicular to the user's arm in the brace, wherein the forearm piece is configured such that the second pin is positioned above the user's 4^{th} metacarpal; wherein a diameter of the first pin opening is approximately 1.5 mm larger than a diameter of the first pin; wherein the palmar piece is moveable relative to the forearm piece when the first pin is installed in the first pin opening and the second pin is installed in the elongated slot, such that the user can move the wrist along the user's dart thrower's plane.

According to an aspect is a brace for a user. The brace includes: (i) a first brace piece configured to conform to a first portion of a user, the first brace piece comprising a first pin extending outwardly from an outer surface of the first brace piece in a first direction, the first brace piece further comprising a second pin extending outwardly from the outer surface of the first brace piece in a first direction, wherein the first direction and the second direction are opposite one another; and (ii) a second brace piece configured to conform to a second portion of a user, the second brace piece comprising a first pin opening sized to receive the first pin, the second brace piece further comprising an elongated slot configured to receive the second pin in a sliding relationship, wherein the elongated slot extends along an axis perpendicular to the first and/or second portion of the user; wherein the first brace piece is moveable relative to the second brace piece when the first pin is installed in the first pin opening and the second pin is installed in the elongated slot.

According to an embodiment, the first and/or second portion of the user is the user's arm or leg.

According to an aspect is a method for applying a brace to a user. The method includes: (i) providing a brace, the brace comprising: a palmar piece configured to conform to the palmar side of a user's wrist, the palmar piece comprising an opening sized for the user's thumb to pass there through, the palmer piece further comprising a first pin extending outwardly from an outer surface of the palmar piece in a first direction, the palmar piece further comprising a second pin extending outwardly from the outer surface of the palmar piece in a first direction, wherein the first direction and the second direction are opposite one another; and a forearm piece configured to conform to the user's forearm, the forearm piece comprising a first pin opening sized to receive the first pin, the forearm piece further comprising an elongated slot configured to receive the second pin in a sliding relationship, wherein the elongated slot extends along an axis perpendicular to the user's arm in the brace; wherein the palmar piece is moveable relative to the forearm piece when the first pin is installed in the first pin opening and the second pin is installed in the elongated slot, such that the user can move the wrist along the user's dart thrower's plane; and (ii) thermoforming the palmar piece and/or the forearm piece to the user.

According to an embodiment, the method further includes the step of 3D printing the palmar piece and the forearm piece.

According to an embodiment, thermoforming the palmar piece and/or the forearm piece to the user comprises heating the palmar piece and/or forearm piece.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description are merely examples of the invention, and are intended to provide an overview or framework for understanding the nature and character of the invention as it is claimed. The accompanying drawings are included to provide a further understanding of the invention, and are incorporated in and constitute a part of this specification. The drawings illustrate various embodiments of the invention and together with the description serve to explain the principles and operation of the invention.

### BRIEF DESCRIPTION OF THE DRAWINGS

The present invention will be more fully understood and appreciated by reading the following Detailed Description in conjunction with the accompanying drawings, in which:

FIG. 1 is a schematic representation of a brace system, in accordance with an embodiment.

FIG. 2 is a schematic representation of a brace system, in accordance with an embodiment.

FIG. 3 is a schematic representation of a brace system, in accordance with an embodiment.

FIG. 4 is a schematic representation of a brace system, in accordance with an embodiment.

FIG. 5 is a schematic representation of a brace system, in accordance with an embodiment.

FIG. 6 is a schematic representation of a brace system, in accordance with an embodiment.

FIG. 7 is a schematic representation of a brace system, in accordance with an embodiment.

FIG. 8 is a schematic representation of a brace method, in accordance with an embodiment.

### DETAILED DESCRIPTION OF EMBODIMENTS

The present disclosure is directed to a wrist brace system. In one embodiment, the system is a wrist brace that allows movement only in the dart thrower's plane (DTP). The wrist brace design is more user friendly and comfortable than existing designs. In function, the brace restricts motion to the DTP while delivering accurate, smooth movement. The brace does not hinder the healing process of the SL ligament or the incision area. Limited motion can improve the healing process for injured ligaments or bones and/or prevent high-risk joint motion.

According to an embodiment, the brace system functions through the use of a mechanism to permit motion aligned with a selected portion of a naturally occurring range of motion. The brace's design is composed of multiple pieces connected with a system of mechanisms to allow for linear sliding of a user's selected joint by combining the motions of rotation and translation. One example of how this mechanism might work is a combined pin and slider system, which provides the user a range of both rotational and translational movement that can be calibrated to accommodate any plane of motion. The dual nature of the pin-slider system allows a non-fixed, floating plane of motion to be created, so the user is not limited to a stationary axis of rotation. While the wrist brace system described or otherwise envisioned herein is designed to restrict motion to the DTP for the use of healing the scapho-lunate ligament, the design can be easily adjusted to accommodate other planes or ranges of motion.

According to an embodiment, the brace system comprises two or more independent pieces joined together by a mechanism such as the pin-slider system. The pieces are made to support members of the body located in close proximity on either side of the joint of interest. For application at the wrist, one piece supports the hand while the second is made for the forearm. According to an embodiment, the pieces are 3D printed and assembled before being thermo-molded to the user. The brace can be designed to provide optimal support and restrict motion while still allowing comfort and breathability.

According to an embodiment, one arrangement of the pins and slider system is designed with the intent of limiting the motion of a wrist to the DTP. The pins allow for rotation, or radial/ulnar deviation, while the slider allows for a slight amount of linear flexion/extension in the wrist. According to an embodiment, the alignment of the pins and slider can be modified to shift the allowable motion to various desired planes of motion.

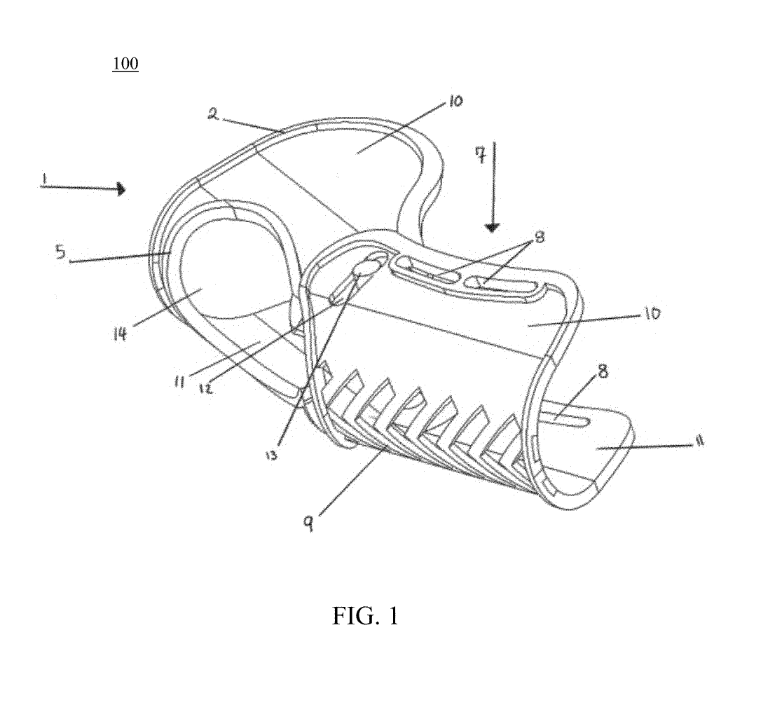

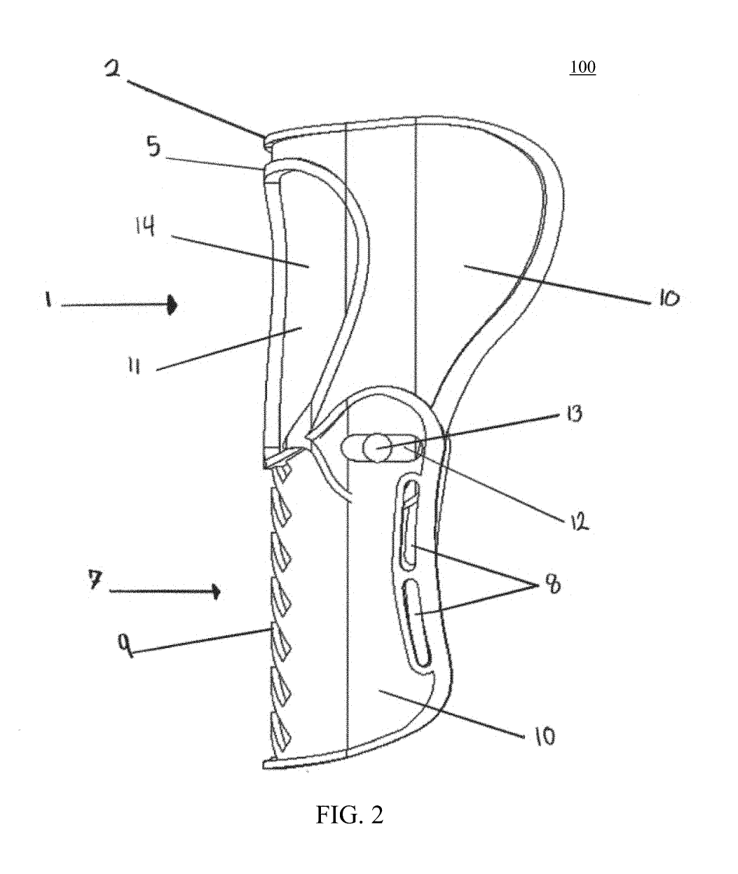

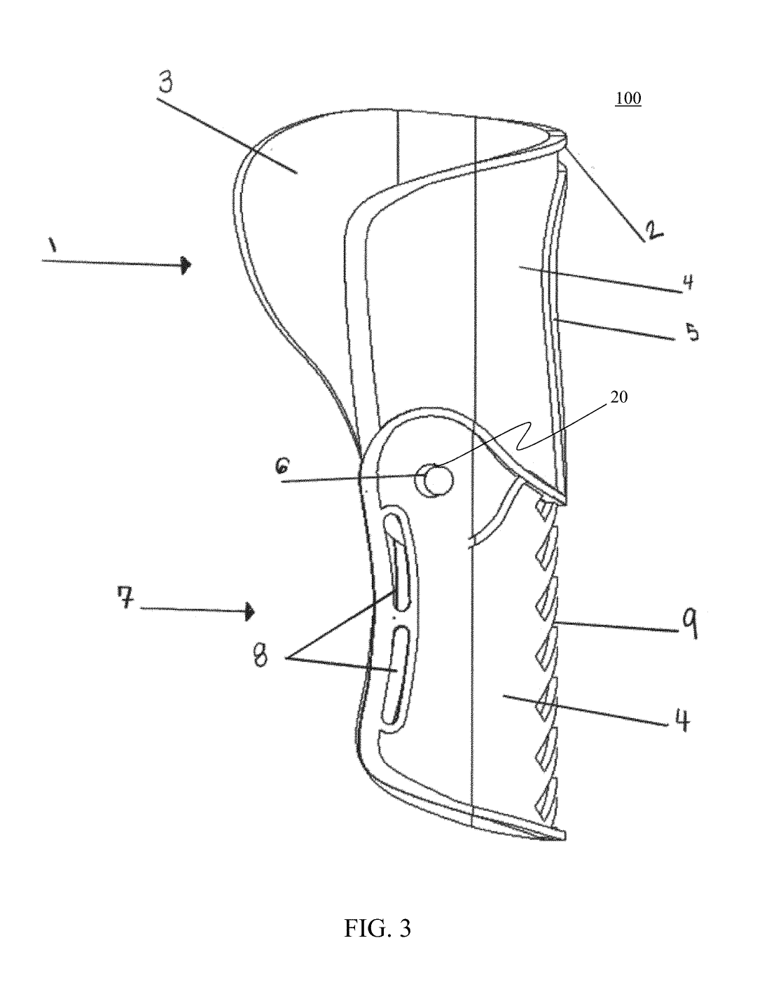

Referring to FIG. 1, in one embodiment, is an isometric view of an assembled and molded wrist brace 100. FIG. 1 provides a view of the dorsal side of the wrist brace, which contains the sliding mechanism. FIG. 1 also provides a view of the lateral side of the brace, which contains the thumb hole and has been fitted with a mesh base. Referring to FIG. 2, in one embodiment, is an assembled brace 100 shown from the dorsal side of the wrist. The dorsal side contains a slider system, which provides a linear range of motion in the wrist. Referring to FIG. 3, in one embodiment, is the palmar side of the brace 100, which contains the second pin of the rotational pin system. This view more clearly shows the final part of the mechanisms in the brace, which allows for the unique plane of motion.

Accordingly, referring to FIGS. 1-3 is one embodiment of a brace system 100. The wrist brace comprises at least two pieces, namely a distal, palmar piece 1 and a proximal, forearm piece 7. According to an embodiment, outer edge 2 of the brace is thickened for rigidity and strength. The brace comprises a thumb hole 14, which comprises a lip 5 which thickened for rigidity and strength. The inside surfaces of the brace are labeled 11 and 3 on the dorsal and palmar sides, respectively. The outside surfaces of the palmar and dorsal sides are 4 and 10, respectively.

According to an embodiment, the brace comprises meshing 9 along the lateral side of the forearm brace piece. Meshing may make molding easier by reducing material, while also increasing breathability and maintaining support. The brace can be attached to the arm with straps (not shown) that go through slots 8 along both sides of the brace.

According to an embodiment, distal palmar piece 1 and proximal forearm piece 7 are connected using palmar pin 6 and dorsal pin 13. Referring to FIG. 3, for example, pin 6 is integral to palmar piece 1, while forearm piece 7 comprises an opening 20 into which pin 6 fits. Opening 20 can be sized, and/or pin 6 can comprise a length, such that pin 6 fits snugly into opening 20 to allow movement but prevents the pin from easily withdrawing from opening 20. Similarly, referring to FIG. 2, for example, pin 13 is integral to palmar piece 1, while forearm piece 7 comprises a slot 12 into which pin 13 fits. Opening 12 can be sized, and/or pin 13 can comprise a length, such that pin 13 fits snugly into opening 12 to allow movement but prevents the pin from easily withdrawing from opening 12.

According to one embodiment, opening 20 is larger in diameter than pin 6. For example, opening 20 may be approximately 1.5 mm larger in diameter than the pin. This allows the palmer pin 6 and the axis of rotation to shift slightly in conjunction with movement of the dorsal pin 13 in opening 12. For the DTP embodiment, palmar pin 6 is placed above the scaphoid tubercle when worn by the user, and the dorsal pin 13 is located over the dorsal surface of the base of the 4th metacarpal when worn by the user. Also for the DTP embodiment, slot 12 is vertical along the 4th metacarpal, which allows the wrist to rotate through the full oblique DTP while preventing other undesirable movement. To ensure that radial-ulnar deviation (RUD) is limited within the DTP, a stopping mechanism (not shown) can be implemented. The stopping mechanism can also limit other forms of movement, such as flexion-extension and/or other movements.

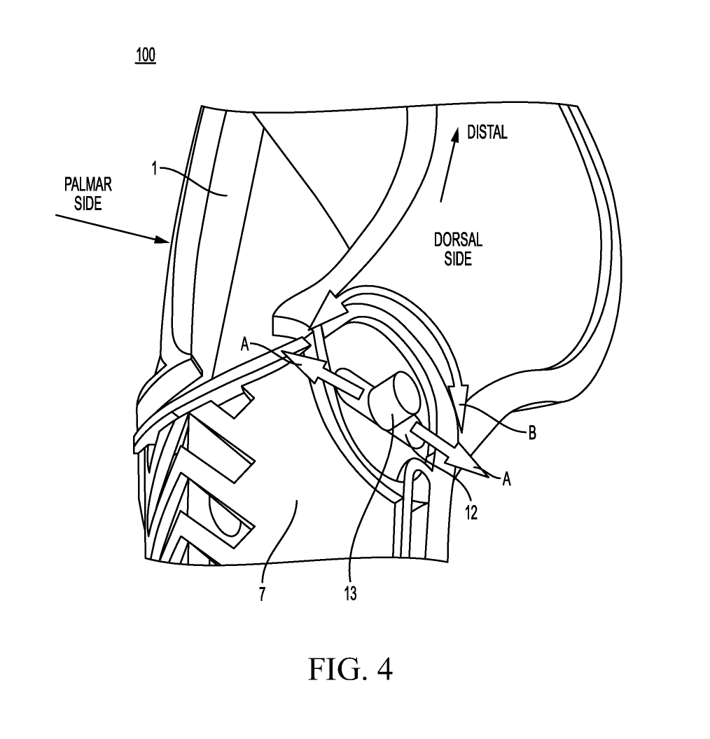

Referring to FIG. 4, in one embodiment, is a schematic representation of a side view of the brace system 100. The brace comprises a distal palmar piece 1 and proximal forearm piece 7. Pin 13 of palmar piece 1 is inserted into slot 12 of forearm piece 7 to allow movement of pin 13 along axis A-A. Similarly, pins 6 and 13 allow rotation of the two pieces (palmar piece 1 and forearm piece 7) relative to each other along the axis of rotation B shown in FIG. 4.

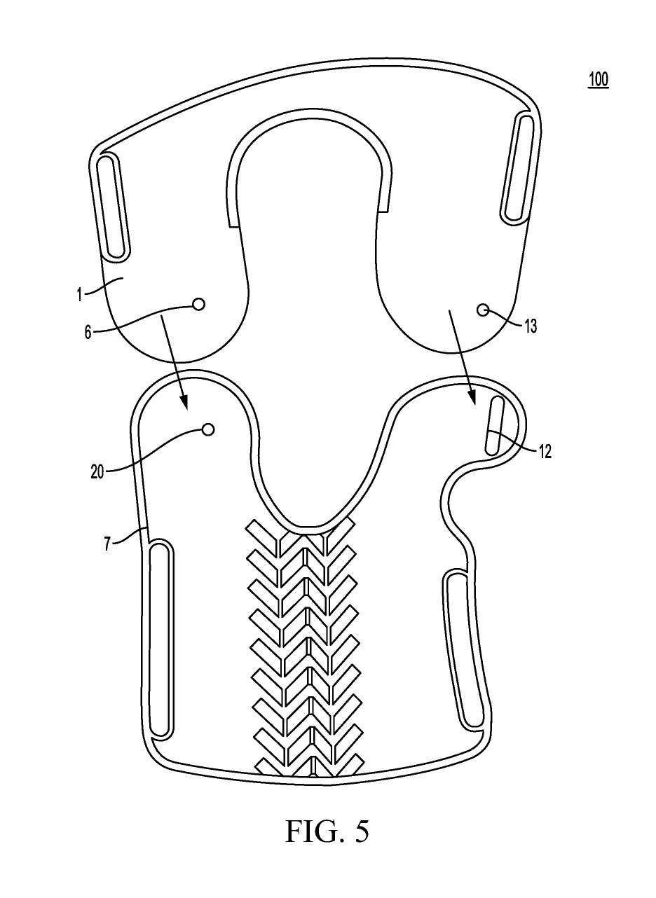

Referring to FIG. 5, in one embodiment, is a schematic representation of brace system 100 with distal palmar piece 1 and proximal forearm piece 7 flattened to show the various elements of the system. Palmar piece 1 comprises pins 6 and 13 (extending into the page in this view), although the pins may be integral to palmar piece 1 and therefore normally not visible from reverse side as in this view. Forearm piece 7 comprises opening 20 for pin 6 and slot 12 for pin 13.

According to an embodiment, slot 12 and pin 13 form a slider mechanism at the joint that creates a translating and/or floating axis of rotation. In the dart-thrower's example, the dorsal slider allows translational movement. This slider allows for a small range of the flexion/extension motion of the wrist with the neutral position of the pin at the center of the slider. On the palmar side, according to an embodiment, the pinhole is slightly larger in diameter than the pin. This tolerance allows for the palmar pin and axis of rotation to shift slightly in conjunction with the dorsal slider. These considerations allow the wrist to rotate through the full DTP.

According to an embodiment, the brace provides support to the area of concern and restricts harmful movements, while still allowing an optimal range of motion. For example, the design of the brace follows good splinting practices by supporting two-thirds of the forearm and avoids interference with any anatomical landmarks such as the ulnar styloid. Among other design improvements, the hand piece is entirely proximal to the transverse palmar crease, permitting unrestricted phalangeal movement; a mesh design simplifies molding and allows superior breathability; the brace is custom fit to the patient so the mechanisms are in the correct locations; to ensure the rigidity and conformity of the brace, thickened segments are created along the palmar and dorsal sides of the forearm and hand; and a strap system ensures a snug fit while making it easy don and doff the brace.

According to an embodiment, the wrist brace described or otherwise envisioned herein constrains motion to the DTP due to the unique pin and slider combination. By combining mechanisms of motion, such as incorporating a slot into a dual pin rotational axis, a new type of movement is created. As the pin shifts through the slider, the axis of rotation shifts as well. The combination of these two mechanisms can make any desired plane with subtle alterations to the placements and orientation of the pins and slider. The combination of pins and a slider provide a unique ability to move in both a rotational and translational path. By creating different pin-slider combinations one can feasibly create countless unique planes of motion. The orientation of the mechanisms can be adjusted to accommodate many planes of motion, not just the DTP. Several ways this modification could be done are by moving pin locations, adding a slider on one or both pins, changing the direction of the slider, or having the pin hole be a clearance fit adding some flexibility in the pin's alignment and therefore axis of rotation. An example of this is found in the PMB for the DTP. The proposed design provides the necessary support of a brace while allowing some motion in a restricted plane to encourage healing while enabling appropriate levels of motion.

According to an embodiment, to prevent an abundance of radial/ulnar deviation, a stopping mechanism may be implemented on the outside of the brace. The stopping mechanism will not inhibit motion in the desired plane but will create an interference to movement that is past the level of safety. This mechanism will allow partial movement in some planes while preventing the motion from being so extreme as to create injury. The stopping mechanism can also limit other forms of movement, such as flexion-extension and/or other movements.

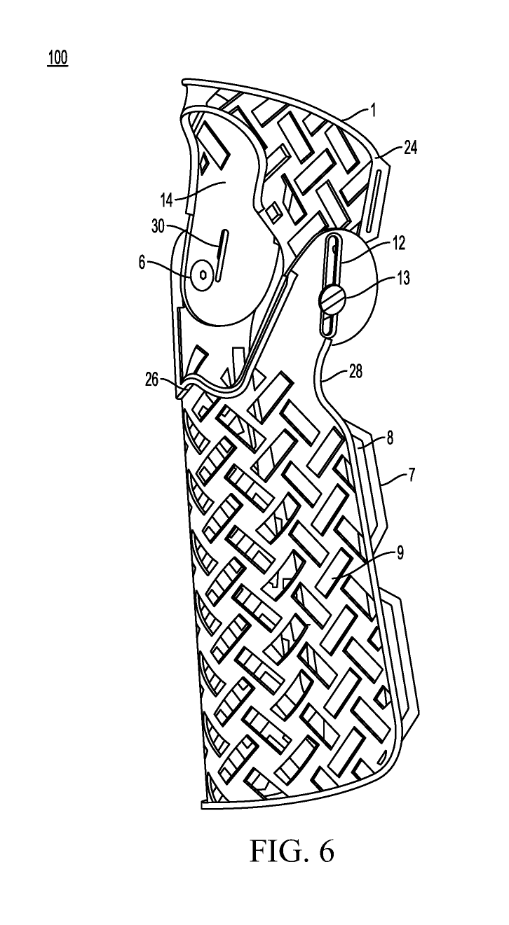

Referring to FIG. 6, in one embodiment, is a schematic representation of brace system 100 with distal palmar piece 1 and proximal forearm piece 7. According to an embodiment, one or more edges 24 of the brace are thickened for rigidity and strength. The brace comprises a thumb hole 14, which can comprise a lip thickened for rigidity and strength. One or more portions of the brace may comprise meshing 9 which can make molding easier by reducing material, while also increasing breathability and maintaining support. The brace can be attached to the arm with straps (not shown) that go through slots 8 along both sides of the brace.

The brace may also comprise a radius flare 26 which reduces pressure over the radius. The brace may also comprise a cutaway or notch 28 which can prevent interference with the ulnar-styloid. The brace may also comprise a palmar slot 30 which allows adjustment of the stopping mechanism, such as shifting of the stopping mechanism which limits radial-ulnar deviation (RUD) (and/or other movements).

According to an embodiment, distal palmar piece 1 and proximal forearm piece 7 are connected using palmar pin 6 and dorsal pin 13. Referring to FIG. 6, for example, pin 6 is integral to palmar piece 1, while forearm piece 7 comprises an opening (not shown) into which pin 6 fits. Similarly, pin 13 is integral to palmar piece 1, while forearm piece 7 comprises a slot 12 into which pin 13 fits. Opening 12 can be sized, and/or pin 13 can comprise a length, such that pin 13 fits snugly into opening 12 to allow movement but prevents the pin from easily withdrawing from opening 12. In FIG. 6, for example, slot 12 extends up and down relative to the arm or limb, while in other embodiments slot 12 extends substantially perpendicular relative to the arm or limb (such as the embodiment depicted in FIG. 1).

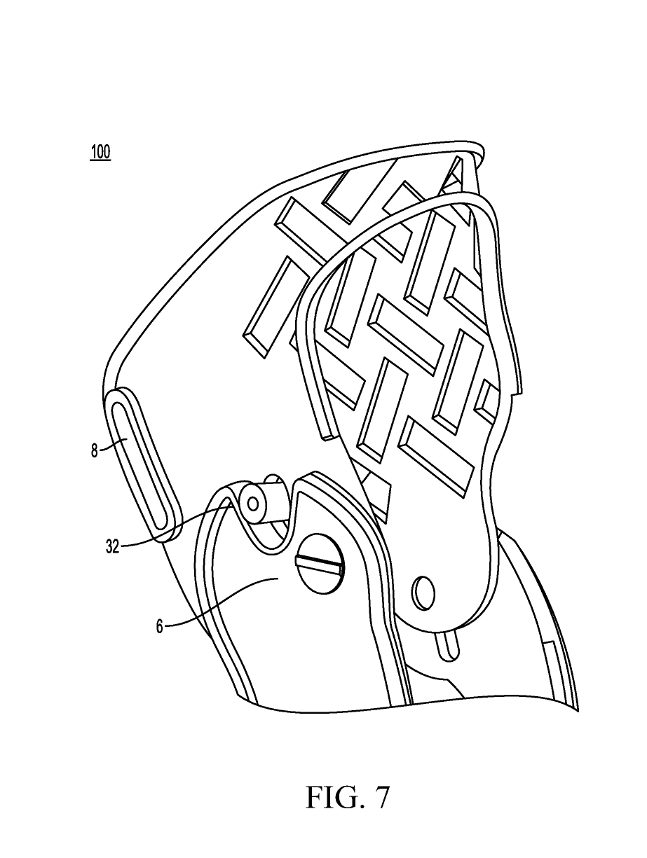

Referring to FIG. 7, in one embodiment, is a schematic representation of a portion of brace system 100. The brace comprises a stopping mechanism 32, composed of a notch in forearm piece 7 and a pin in palmar piece 1.

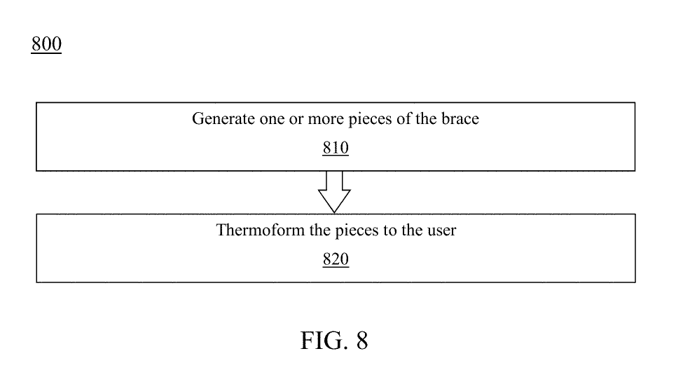

Referring to FIG. 8 is an embodiment of a method 800 for applying a brace 100 to a user. At step 610 of the method, one or more pieces of the brace are generated. The pieces may be printed using a 3D printer, and/or created via a molding process, and/or generated via any other system or process. According to an embodiment, the pieces may be composed of any material suitable for use in a brace, which may include one or more compounds known to be suitable for use in a brace. Among many other compounds, the brace may be composed in part or in whole of polylactic acid (PLA) printer filament, polyethylene terephthalate (PETG), and/or any other compound.

At step 820 of the method, the two or more pieces are thermoformed to ensure a custom fit to the user. The pieces can be thermoformed in a practitioner's office, a hospital room, or any other clinical or typical office for brace fitting. According to an embodiment, the thermoforming process comprises aligning the pins by placing them over anatomical landmarks while thermo-molding, which establishes the axis of rotation for the DTP. The brace is heated in a hot water bath (such as between 195° F. and 200° F., although the temperature may depend on the material) and then molded to the patient. The heating and molding process is repeated as needed to custom fit the brace to each individual.

The brace system 100 described or otherwise envisioned herein provides an innovative design that augments wrist injury rehabilitation by virtue of precise, limited motion. According to an embodiment, the brace system can be adapted to activities that subject the brace to intense stresses and strains such as heavy lifting, contact sports, or high-impact activities. The brace can also be adapted to accommodate other common carpal injuries, such as by making changes to the design to allow movements about different axes of motion. Using the brace, post-surgery patients regain limited mobility of their wrist faster than traditional rigid braces, offering improved patient quality of life by enabling some normality in basic functions such as drinking from a mug, cutting with a knife, or tying shoes. Returning to daily activities earlier during the recovery process can prevent compensatory injuries due to coping strategies while improving patient quality of life. The brace system also allows wrist motion in the DTP, ultimately encouraging healing while improving quality of life during recovery.

While embodiments of the present invention has been particularly shown and described with reference to certain exemplary embodiments, it will be understood by one skilled in the art that various changes in detail may be effected therein without departing from the spirit and scope of the invention as defined by claims that can be supported by the written description and drawings. Further, where exemplary embodiments are described with reference to a certain number of elements it will be understood that the exemplary embodiments can be practiced utilizing either less than or more than the certain number of elements.

What is claimed is:

1. A brace, comprising:
a palmar piece configured to conform to the palmar side of a user's wrist, the palmar piece comprising an opening sized for the user's thumb to pass there through, the palmer piece further comprising a first pin extending outwardly from an outer surface of the palmar piece in a first direction, the palmar piece further comprising a second pin extending outwardly from the outer surface of the palmar piece in a first direction, wherein the first direction and the second direction are opposite one another; and
a forearm piece configured to conform to the user's forearm, the forearm piece, the forearm piece comprising a first pin opening sized to receive the first pin, the forearm piece further comprising an elongated slot configured to receive the second pin in a sliding relationship, wherein the elongated slot extends along an axis perpendicular to the user's arm in the brace;
wherein the palmar piece is moveable relative to the forearm piece when the first pin is installed in the first pin opening and the second pin is installed in the elongated slot, such that the user can move the wrist along the user's dart thrower's plane.

2. The brace of claim 1, wherein the palmar piece is configured such that the first pin is positioned about the user's scaphoid tubercle.

3. The brace of claim 1, wherein the forearm piece is configured such that the second pin is positioned above the user's 4^{th} metacarpal.

4. The brace of claim 1, wherein a diameter of the first pin opening is larger than a diameter of the first pin.

5. The brace of claim 5, wherein the diameter of the first pin opening is approximately 1.5 mm larger than the diameter of the first pin.

6. The brace of claim 1, wherein at least a portion of an outer edge of the brace is thicker than an inner portion of the brace.

7. The brace of claim 1, wherein at least a portion of an outer edge of the thumb opening is thicker than an inner portion of the brace

8. The brace of claim 1, wherein at least a portion of the forearm piece comprises meshing.

9. The brace of claim 1, wherein the forearm piece comprises two or more slots configured to receive an attachment mechanism around the dorsal portion of the user's forearm.

10. The brace of claim 1, further comprising a stopping mechanism configured to limit a movement of the user's wrist.

11. A brace, comprising:
a palmar piece configured to conform to the palmar side of a user's wrist, the palmar piece comprising an opening sized for the user's thumb to pass there through, the palmer piece further comprising a first pin extending outwardly from an outer surface of the palmar piece in a first direction, the palmar piece further comprising a second pin extending outwardly from the outer surface of the palmar piece in a first direction, wherein the first direction and the second direction are opposite one another, further wherein the palmar piece is configured such that the first pin is positioned about the user's scaphoid tubercle; and
a forearm piece configured to conform to the user's forearm, the forearm piece comprising a first pin opening sized to receive the first pin, the forearm piece further comprising an elongated slot configured to receive the second pin in a sliding relationship, wherein the elongated slot extends along an axis perpendicular to the user's arm in the brace, wherein the forearm piece is configured such that the second pin is positioned above the user's 4^{th} metacarpal;
wherein a diameter of the first pin opening is approximately 1.5 mm larger than a diameter of the first pin;
wherein the palmar piece is moveable relative to the forearm piece when the first pin is installed in the first pin opening and the second pin is installed in the elongated slot, such that the user can move the wrist along the user's dart thrower's plane.

12. The brace of claim 11, wherein at least a portion of an outer edge of the brace is thicker than an inner portion of the brace

13. The brace of claim 11, wherein at least a portion of an outer edge of the thumb opening is thicker than an inner portion of the brace

14. The brace of claim 11, wherein the forearm piece comprises two or more slots configured to receive an attachment mechanism around the dorsal portion of the user's forearm.

15. The brace of claim 11, further comprising a stopping mechanism configured to limit a movement of the user's wrist.

16. A brace, comprising:
a first brace piece configured to conform to a first portion of a user, the first brace piece comprising a first pin extending outwardly from an outer surface of the first brace piece in a first direction, the first brace piece further comprising a second pin extending outwardly from the outer surface of the first brace piece in a first direction, wherein the first direction and the second direction are opposite one another; and
a second brace piece configured to conform to a second portion of a user, the second brace piece comprising a first pin opening sized to receive the first pin, the second brace piece further comprising an elongated slot configured to receive the second pin in a sliding relationship, wherein the elongated slot extends along an axis perpendicular to the first and/or second portion of the user;
wherein the first brace piece is moveable relative to the second brace piece when the first pin is installed in the first pin opening and the second pin is installed in the elongated slot.

17. The brace of claim 16, wherein the first and/or second portion of the user is the user's arm or leg.

18. A method for applying a brace to a user, comprising the steps of:
providing a brace, the brace comprising: (i) a palmar piece configured to conform to the palmar side of a user's wrist, the palmar piece comprising an opening sized for the user's thumb to pass there through, the palmer piece further comprising a first pin extending outwardly from an outer surface of the palmar piece in a first direction, the palmar piece further comprising a second pin extending outwardly from the outer surface of the palmar piece in a first direction, wherein the first direction and the second direction are opposite one another; and (ii) a forearm piece configured to conform to the user's forearm, the forearm piece comprising a first pin opening sized to receive the first pin, the forearm piece further comprising an elongated slot configured to receive the second pin in a sliding relationship, wherein the elongated slot extends along an axis perpendicular to the user's arm in the brace; wherein the palmar piece is moveable relative to the forearm piece when the first pin is installed in the first pin opening and the second pin is installed in the elongated slot, such that the user can move the wrist along the user's dart thrower's plane; and
thermoforming the palmar piece and/or the forearm piece to the user.

19. The method of claim 18, further comprising the step of 3D printing the palmar piece and the forearm piece.

20. The method of claim 18, wherein thermoforming the palmar piece and/or the forearm piece to the user comprises heating the palmar piece and/or forearm piece.
